(19) United States
(12) Patent Application Publication
(10) Pub. No.: US 2016/0365011 A1
(43) Pub. Date: Dec. 15, 2016

(54) COMBINATION WRISTBAND AND LABEL FORM

(71) Applicant: WARD KRAFT, INC., FORT SCOTT, KS (US)

(72) Inventors: ROGER DAVIS, GARLAND, KS (US); GINA HOLT, FORT SCOTT, KS (US)

(21) Appl. No.: 15/180,593

(22) Filed: Jun. 13, 2016

Related U.S. Application Data
(60) Provisional application No. 62/175,055, filed on Jun. 12, 2015.
(60) Provisional application No. 62/247,863, filed on Oct. 29, 2015.
(60) Provisional application No. 62/256,465, filed on Nov. 17, 2015.

(51) Int. Cl. G09F 3/00; A61B 90/90; G09F 3/10

(52) U.S. Cl. CPC G09F 3/005; G09F 3/10; G09F 3/0288; A61B 90/90; G09F 2003/0201

(57) ABSTRACT

A combination wristband and label form comprises a top portion and a bottom portion. The top portion has a plurality of labels die cut therein. Each of the labels is configured to be removable from the form. The bottom portion has a wristband configured to receive an indicia, and an outer portion surrounding the wristband. The wristband is separable from the form and is formed of a single ply of water-resistant and tamper-resistant material. The wristband has a first lateral end and a second lateral end. The first lateral end has an adhesive area comprising adhesive adjacent the first lateral end. The second lateral end comprises perforations. The form includes a release liner having a release material. Removal of the wristband from the form exposes a first area of the release liner. The form is configured for multiple passes through a printer after the wristband is removed therefrom.

### RELATED APPLICATIONS

This application claims priority to U.S. Provisional Application No. 62/175,055, filed on Jun. 12, 2015, U.S. Provisional Application No. 62/247,863, filed on Oct. 29, 2015, and U.S. Provisional Application No. 62/256,465, filed on Nov. 17, 2015, the disclosures of each of which are incorporated by reference in their entireties herein.

### BACKGROUND

The wristband is a frequently-used instrument for distinguishing among various groups of people. For example, wristbands may be used to identify persons in short term healthcare facilities, or to distinguish between levels of access (e.g., at a concert) or permissions. Thus, there is a significant market for wristbands. Wristbands with improved performance capabilities are desirable.

Many wristband designs require multiple steps to remove the wristband from its liner and subsequently affix it to the wearer. For example, the user may be required to remove the liner in order to expose adhesive, or to fold a part of the wristband over the face portion in order to secure the wristband in place. Some wristbands include adhesive on both ends thereof, which may make it difficult for the user to affix the wristband without attaching the adhesive to unattended areas. Additionally, this may make it difficult to remove the wristband when desired.

### SUMMARY

The following presents a simplified summary of the invention in order to provide a basic understanding of some aspects of the invention. This summary is not an extensive overview of the invention. It is not intended to identify critical elements of the invention or to limit the scope of the invention. Its sole purpose is to present some concepts of the invention in a simplified form as a prelude to the more detailed description presented below.

According to an embodiment, a combination wristband and label form comprises a top portion and a bottom portion. The top portion has a plurality of labels die cut therein. The bottom portion has a rectangular wristband configured to receive an indicia, and an outer portion surrounding the wristband. The wristband is separable from the form and is formed of a single ply of water-resistant material. The wristband has a first lateral end and a second lateral end. The first lateral end has an adhesive area comprising adhesive adjacent the first lateral end. The second lateral end comprises perforations. There is no adhesive on the wristband outside the adhesive area. The form further comprises a release liner having a release material. Removal of the wristband from the form exposes a first area of the release liner. The first area is devoid of a void space. The outer portion is permanently secured to the release liner.

According to another embodiment, a combination wristband and label form comprises a top portion and a bottom portion. The top portion has a plurality of labels die cut therein. Each of the labels is configured to be removable from the form. The bottom portion has a wristband configured to receive an indicia, and an outer portion surrounding the wristband. The wristband is separable from the form and is formed of a single ply of water-resistant and tamper-resistant material. The wristband has a first lateral end and a second lateral end. The first lateral end has an adhesive area comprising adhesive adjacent the first lateral end. The second lateral end comprises perforations. The form includes a release liner having a release material. Removal of the wristband from the form exposes a first area of the release liner. The form is configured for multiple passes through a printer after the wristband is removed therefrom.

### BRIEF DESCRIPTION OF THE DRAWINGS

FIG. 1 is a front side view of a combination wristband and label form according to one embodiment of the invention.

FIG. 2 is a back side view of the combination wristband and label form according to the embodiment of FIG. 1.

FIG. 3 is a front view of a backing sheet that is attached to a back side of the form of FIG. 1.

FIG. 4 is a front side view of an alternative embodiment of the form of FIG. 1.

FIG. 5 is a front side view of another alternative embodiment of the form of FIG. 1.

### DETAILED DESCRIPTION

Many wristband designs require multiple steps in order to remove the wristband from its liner and subsequently affix it to the wearer. For example, the user may be required to remove the liner in order to expose adhesive, or to fold a part of the wristband over the face portion in order to secure the wristband in place. Some wristbands include adhesive on both ends, which may make it difficult for the user to affix the wristband without attaching the adhesive to unattended areas. Additionally, this may make it difficult to remove the wristband when desired.

Other wristbands may be configured to include two layers of material, fastened together with adhesive. The wristband is thus thicker and heavier. Still further designs include a paper layer which is not water resistant that tends to get torn and tattered. According to these designs, a wristband portion is permanently adhered to a paper backing sheet which is die cut in a form, to form a two-layer wristband. The wristband, consisting of the backing layer and the wristband portion, is removed from the form leaving a wristband-shaped hole in the form. This may be undesirable, because the holes in the form may prevent the form from being able to pass through a printer multiple times.

One embodiment of the present invention, described in detail herein, provides for a wristband which may be removed from a form via one generally continuous motion, and which, upon removal, may be conveniently secured to a wearer as-is. The wristband may have adhesive on one end only. Further, the wristband may be configured to include only a single layer of a light, synthetic (or other similar) material, thus making the wristband approximately half of the thickness of traditional wristbands currently on the market. Finally, the synthetic material may be water and tear resistant such that the wristband will not tear when removed from the backing sheet prior to affixing the wristband to the person. The wristband may be configured to be removed from a form without leaving a hole in the form, thus leaving the backing sheet intact such that the form may be passed through a printer multiple times.

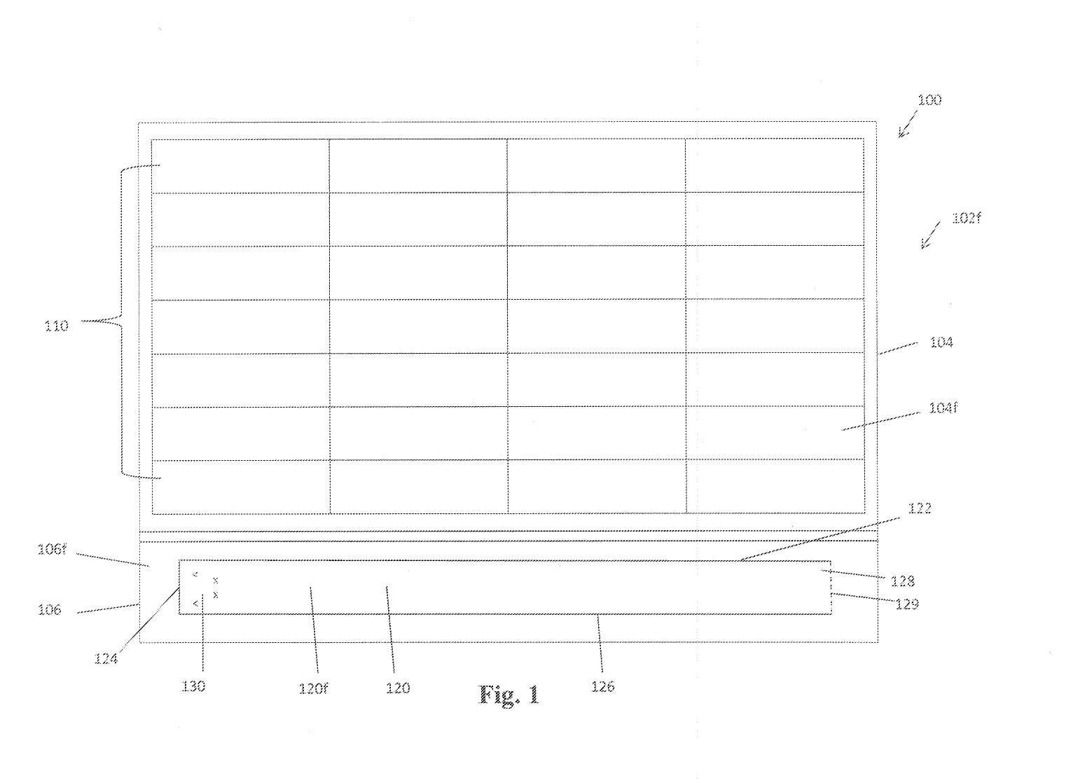

With reference now to the figures, FIG. 1 shows a combination wristband and label form 100 according to one embodiment of the invention. The form 100 includes a front sheet 102 adhered to a backing sheet 140. The front sheet 102 has a front side 102f and a back side 102b. The front sheet 102 may in some embodiments be separated into a top portion 104 and a bottom portion 106 having a relatively small gap therebetween. The top portion 104 and the bottom portion 106 may each have a front face 104f and 106f, respectively.

The front surface 104f of the top portion 104 may include a plurality of labels 110. The labels 110 may be arranged in columns and rows, for example, 4×6. However, the labels 110 may be provided in any combinations of columns (e.g., 1, 2, 3, 4, etc.) and rows (e.g., 1, 2, 3, 4, etc.). The labels 110 may be configured to receive indicia. Accordingly, the front surface 104f may be constructed of paper or other appropriate textile sufficient for receiving ink, e.g., from a printer or other marking device.

The labels 110 may have a variety of constructions. For example, the figures illustrate the labels 110 as having a generally rectangular configuration. However, the labels 110 may be square, circular, polygonal, etc. Additionally, a combination of label configurations may be employed on a single form 100.

The bottom portion 106 may comprise one or more wristbands 120. The wristbands 120 may be configured to be printable. In some embodiments, the form 100 may be configured to be passed through a printer so that indicia (e.g., patient name, patient medications, machine readable information such as barcodes, et cetera) may be printed directly on the wristband 120.

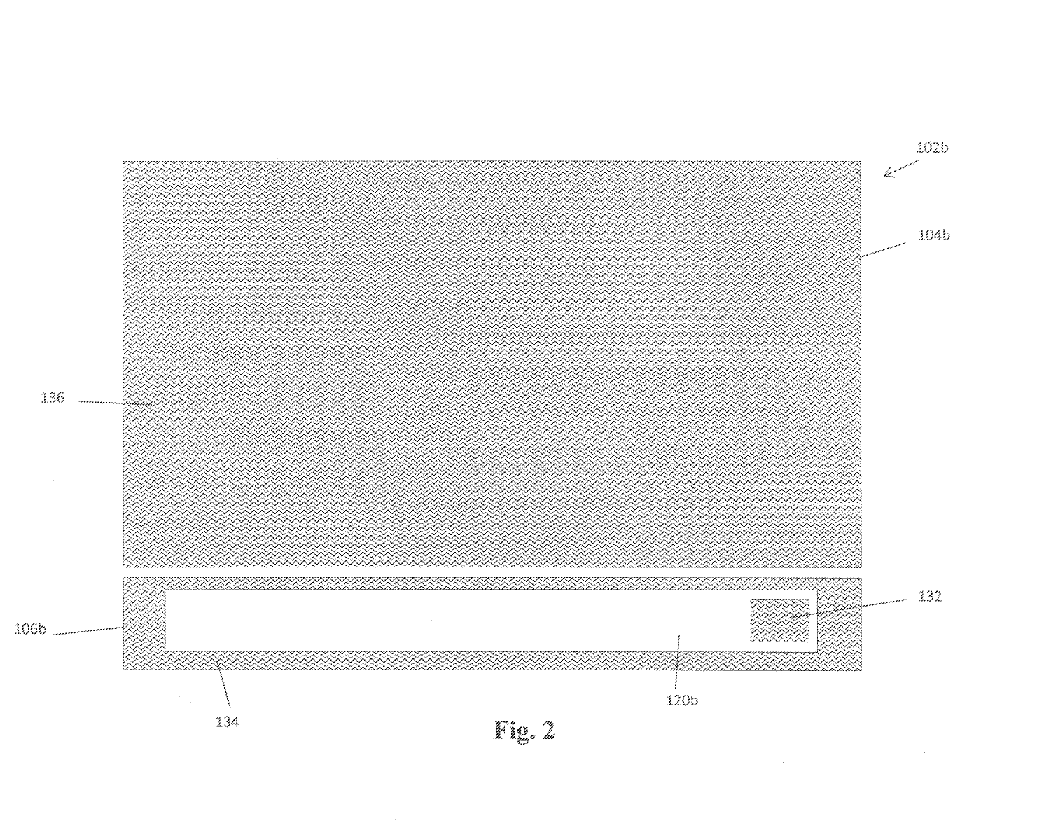

The wristband 120 may be defined by two longitudinally opposing sides (or ends) 122 and 126, which may be die cut into the bottom portion front face 106f, and two laterally opposing sides (or ends) 124 and 128. One of the laterally opposed sides, e.g., side 128, may include perforations 129, and have no adhesive inwardly adjacent the perforations 129. The other laterally opposed side, e.g., side 124, may contain an area of adhesive 132 (FIG. 2) on a backside 120b of the wristband 120. The adhesive 132 may keep the end 124 secured to the backing sheet 140 as described below. The wristband 120 may be substantially held into position via the adhesive patch 132 and the perforated side 128. The wristband 120 may contain no adhesive apart from the adhesive 132 adjacent the end 124.

The wristband 120 may be generally rectangular, as shown. Alternatively, the wristband 120 may take on other desirable shapes. In one embodiment, a height of the adhesive end (e.g., end 124) may be less than a height of the remainder of the wristband 120 (including being less than the height of the end 128).

The wristband 120 may be further equipped with security slits 130. The security slits 130 may be configured to tear, should the wristband 120 be tampered with after the wristband 120 is applied to a wearer. This may be beneficial to ensure that the wristband 120 remains associated with the intended wearer, particularly in a healthcare environment where the wristband 120 includes patient-specific information.

The bottom portion 106 may be constructed of a synthetic material, such as polyester fabric or plastic, for example. Other materials may additionally, or alternately, be appropriate. Those of skill in the art may recognize that it may be beneficial for the wristband 120 material to be resistant to water or other liquid, which may cause the integrity of the wristband 120 to be prematurely compromised.

Attention is now directed to FIG. 2, which shows the back side 102b of the form 100. The back side 102b may include a back face 104b of the top portion 104 and a back face 106b of the bottom portion 106. The back face 104b of the top portion 104 may include an adhesive area 136. The adhesive area 136 may allow for the labels 110 to be releasably secured to the backing sheet 140. The back face 106b of the bottom portion 106 may additionally have adhesive areas 132, described above, and 134. The adhesive area 134 may correspond to the area surrounding the wristband 120 which remains in place when the wristband 120 is removed from the form 100.

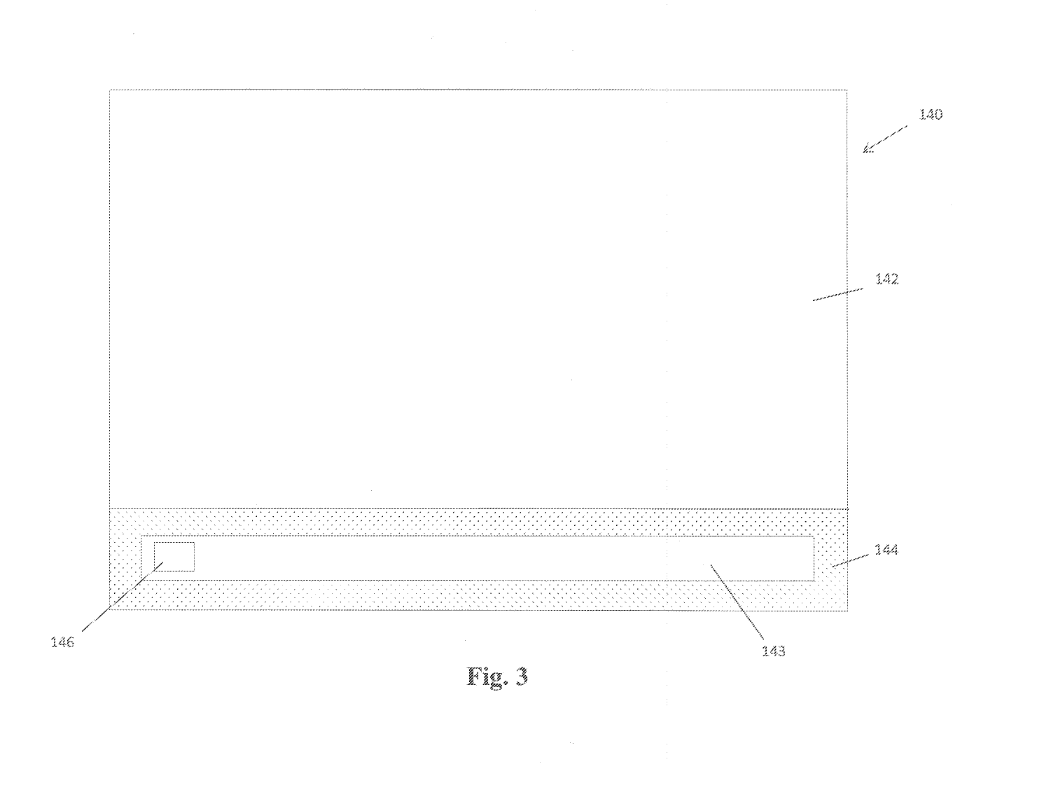

The adhesive areas 132, 134, and 136 of the back faces 104b and 106b may adhere to the backing sheet 140, illustrated in FIG. 3. The backing sheet 140 may be constructed of paper or a synthetic resin, and may include a layer of silicone (or another similar release material) in the areas corresponding to the adhesive areas 132, 134, and 136. For example, the adhesive area 136 may releasably adhere to the silicone material 142, adhesive area 132 may releasably adhere to silicone material 146, and adhesive area 134 may adhere to silicone material 144. The silicone material 144 may be applied in a pattern. The patterned silicone 144 may allow for a more permanent adhesion between the backing sheet 140 and the front sheet 102 in areas void of silicone (e.g., the bond between the area of the bottom portion 106 surrounding the wristband 120 and the backing sheet 140 may be stronger than the bond between the top portion 104 and the backing sheet). This may keep the area of the bottom portion 106 surrounding the wristband 120 in place on the backing sheet 140. In some embodiments, the silicone material 144 may be omitted so that the area of the bottom portion 106 surrounding the wristband 120 permanently adheres to the backing sheet 140.

When the wristband 120 is removed from the form 100, the area 143 of the backing sheet 140 behind the wristband 120 may remain intact. Such may provide several benefits over prior art wristbands. For example, as noted above, prior art methods consisting of “punching out” the wristband from the form leaves a void that may prevent the rest of the form from being used at a later time. However, if the form remains intact, as in the present invention, it may be used multiple times, for example, to print on the labels 110. This may be beneficial because it is often desirable to print the labels 110 at different times (for example, it may be desirable to print new labels 110 to reflect changes made to medications prescribed to a patient during the course of his treatment). A new label 110, such as a label 110 leftover on the form 100, may thus be printed with the new information until all the labels 110 have been used. Of course, the labels 110 may be used for any desirable purposes, such as for labeling patient files and other documents, vials, etc. The labels 110 may all be printed with information in a single pass through the printer, or the form 100 may be passed through the printer multiple times such that the labels 110 are printed as needed.

In use, after the wristband 120 has been printed, the user may peel the side 124 of the wristband 120 up and away from the form 100, inserting his or her finger under the wristband side 126 until the finger exits at side 122. The user may then tear the side 128 along the perforations 129 to free the wristband 120 from the form 100. Alternately, the user may hold the wristband 100, e.g., from side 122 or 126, between his index finger and thumb, tear the side 128 along the perforations 129, and then separate the wristband 120, including the side 124 having the adhesive 132, from the form 100. In this way, the user may remove the wristband 120 from the form 100 in one generally continuous motion. The user may then attach the wristband 120 to a person's wrist by wrapping the wristband 120 around the wrist, face up, and fastening the adhesive end (e.g., side 124) to the face of the wristband 120. Such quick and convenient removal of the wristband 120 from the form 100 and its ready securement to a person's wrist may be preferable, as compared for example, to wristbands that must be removed from the associated forms in several steps or which need to be folded or otherwise reconfigured after they have been removed from the form and before they are secured to a wearer's wrist.

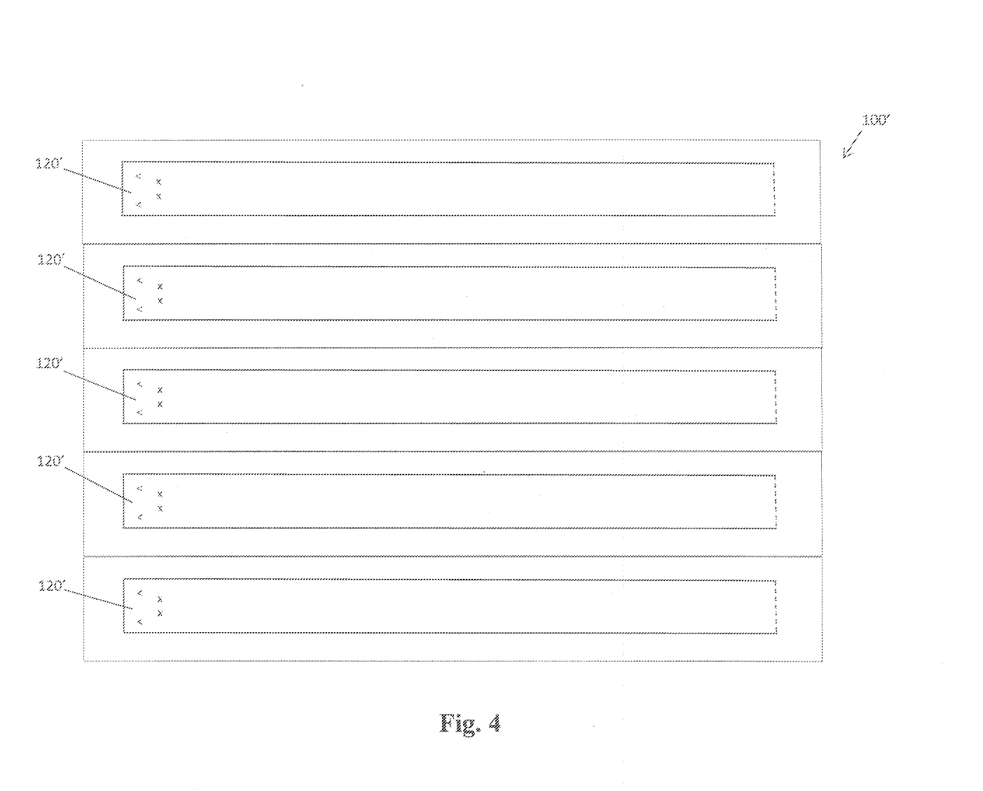

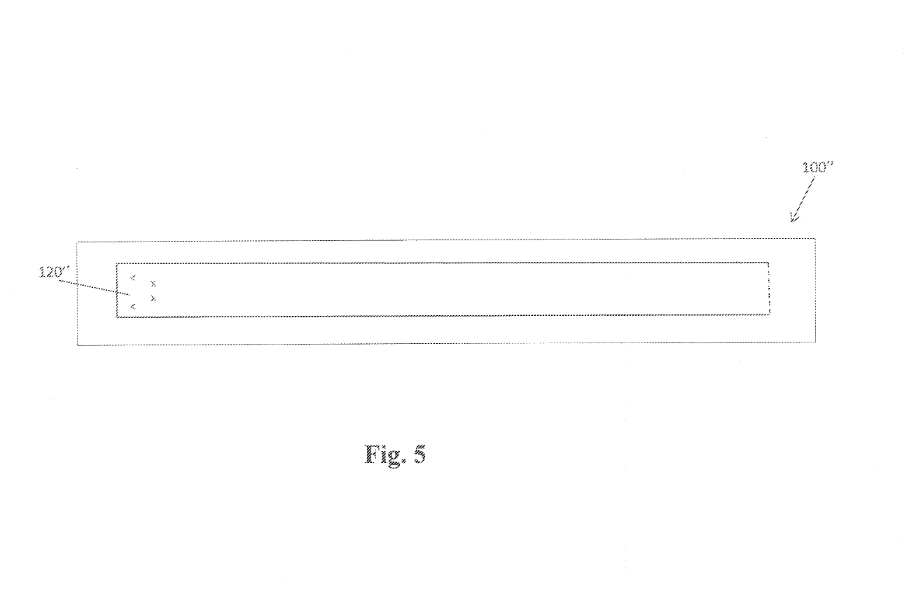

In another embodiment, illustrated in FIG. 4, the form 100′ may consist of a plurality of wristbands 120′ and does not include labels 110. Alternately, the form 100″ may include only a single wristband 120″ as shown in FIG. 5. The form 100 may be approximately the size of a standard piece of paper (e.g., 8½″×11″), or the form 100 may be tailored to the size of the required wristbands 120 and/or labels 110. For example, if only a single wristband 120 is required, the form 100 may be only the size necessary to contain one wristband 120.

Many different arrangements of the described invention are possible without departing from the spirit and scope of the present invention. Embodiments of the present invention are described herein with the intent to be illustrative rather than restrictive. Alternative embodiments will become apparent to those skilled in the art that do not depart from its scope. A skilled artisan may develop alternative means of implementing the disclosed improvements without departing from the scope of the present invention.

Further, it will be understood that certain features and subcombinations are of utility and may be employed without reference to other features and subcombinations and are contemplated within the scope of the claims. Not all steps listed in the various figures and description need to be carried out in the specific order described. The description should not be restricted to the specific described embodiments.

1. A combination wristband and label form, comprising:
a top portion having a plurality of labels die cut therein;
a bottom portion having a rectangular wristband configured to receive an indicia, and an outer portion surrounding the wristband; the wristband being separable from the form and being formed of a single ply of water-resistant material; the wristband having a first lateral end and a second lateral end; the first lateral end having an adhesive area comprising adhesive adjacent thereto; the second lateral end comprising perforations; there being no adhesive on the wristband outside the adhesive area; and
a release liner having a release material;
wherein:
removal of the wristband from the form exposes a first area of the release liner; the first area being devoid of a void space; and
the outer portion is permanently secured to the release liner.

2. The combination wristband and label form of claim 1, wherein the form is configured for multiple passes through a printer after the wristband is removed therefrom.

3. The combination wristband and label form of claim 2, wherein at least one of the first and the second lateral ends is equipped with tamper evident slits.

4. The combination wristband and label form of claim 3, wherein the wristband comprises two longitudinal edges; each of the longitudinal edges and the first lateral end being defined by a die cut in the bottom portion.

5. The combination wristband and label form of claim 4, wherein the wristband is removable from the form in one continuous motion.

6. The wristband and label form of claim 4, wherein each of the longitudinal edges define a gap between the wristband and the release liner.

7. A combination wristband and label form, comprising:
a top portion having a plurality of labels die cut therein; each of said labels being configured to be removable from the form;
a bottom portion having a wristband configured to receive an indicia, and an outer portion surrounding the wristband; the wristband being separable from the form and being formed of a single ply of water-resistant and tamper-resistant material;
the wristband having a first lateral end and a second lateral end; the first lateral end having an adhesive area comprising adhesive adjacent thereto; the second lateral end comprising perforations;
a release liner having a release material;
wherein:
removal of the wristband from the form exposes a first area of the release liner;
the form is configured for multiple passes through a printer after the wristband is removed therefrom.

8. The combination wristband and label form of claim 7, wherein the wristband is generally rectangular.

9. The combination wristband and label form of claim 8, wherein a height of the first lateral end is less than a height of the second lateral end.

10. The combination wristband and label form of claim 9, wherein at least one of the plurality of said labels has a different shape than another of the plurality of said labels.

11. The combination wristband and label form of claim 10, wherein at least one of the first and the second lateral ends is equipped with tamper evident slits.

12. The combination wristband and label form of claim 11, wherein the first longitudinal end is equipped with tamper evident slits.

13. The combination wristband and label form of claim 11, wherein there is a gap between the top portion and the bottom portion.

14. The combination wristband and label form of claim 13, wherein the release material is silicone.
